(19) United States
(12) Patent Application Publication
(10) Pub. No.: US 2021/0049147 A1
(43) Pub. Date: Feb. 18, 2021

(54) PROCESS MODEL REPAIRING METHOD BASED ON STRUCTURE REPLACEMENT

(71) Applicant: SHANDONG UNIVERSITY OF SCIENCE AND TECHNOLOGY

(72) Inventors: Yuyue DU, Qingdao (CN); Yuhua XU, Qingdao (CN); Wenjing LUAN, Qingdao (CN); Liang QI, Qingdao (CN); Fuxin ZHANG, Qingdao (CN); Lu WANG, Qingdao (CN); Yinhua TIAN, Qingdao (CN)

(21) Appl. No.: 16/962,189

(22) PCT Filed: Sep. 26, 2019
(86) PCT No.: PCT/CN2019/108010

§ 371 Date: Jul. 14, 2020

(30) Foreign Application Priority Data
Apr. 17, 2019 (CN) 201910306217.8

(51) Int. Cl. G06F 16/23; G06Q 10/10; G06Q 10/06; G06Q 30/00; G06Q 10/00; G06F 17/11

(52) U.S. Cl. CPC G06F 16/2365; G06Q 10/10; G06Q 10/067; G06Q 30/018; G16H 40/20; G06Q 10/06395; G06F 16/2379; G06F 17/11; G06Q 10/20

(57) ABSTRACT

The present disclosure provides a process model repairing method based on structure replacement. Firstly, two ordering relationship sets of an event log and a model are obtained by re-defining ordering relationships between activities, and a deviation set recording differences between the event log and the model is obtained; next, the model is repaired according to the deviation set. A selective structure or a sequential structure is changed into a concurrent structure according to the given event log in the present disclosure. The repaired model indicated by a logical Petri net is capable of accurately reflecting a logical relationship between activities. The method of the present disclosure and other model repairing methods are compared through simulation experiments. Experimental results show that a process model repaired by the method of the present disclosure has higher precision, correctly reflects an actual business process, and improves an execution efficiency of the business process, and the model indicated by the logical Petri net has better simplicity.

### TECHNICAL FIELD

The present disclosure relates to a process model repairing method based on structure replacement.

### BACKGROUND

In recent years enterprise information systems become increasingly important in realizing business processes. As the enterprise information systems are widely applied, a great number of event logs are generated. The event log includes a plurality of traces, and each trace corresponds to one execution of the business process. Process mining technology may discover, test, verify and improve an actual business process according to the event logs. Process mining mainly has three functions: process discovery, conformance checking and process enhancement. The process discovery may construct a corresponding process model for a common business process according to the event logs generated in the enterprise information system. In recent years, many process mining algorithms are proposed. For example, an algorithm α designed by Aalst et al. may construct a model according to an ordering relationship between activities, but cannot effectively obtain a model containing repetitive activities, invisible conversion or some complex structures. Thus, many variations of the algorithm α appear. The quality of a process model is mainly measured by the following four indicators: fitness, simplicity, precision, and generalization. The fitness, as the most important indicator, means that the model can replay all traces in the event log; the precision refers to that the model does not allow unobservable behaviors to appear in the event log; the simplicity requires the model capable of replaying the event log to be as simple as possible; and the generalization refers to that the model is not limited to behaviors seen in the log. The conformance checking can discover differences between logs by replaying the logs on the model. The existing conformance checking methods mainly include alignment, token replay and footprint comparison. The process enhancement takes the event log and the process model as input data, with its output being an extended model. When the business process changes over time and its corresponding process model is not yet updated, a new model may be mined by using a process discovery algorithm. However, similarity between the new model and the original model is often low. Therefore, a better method is to repair the original model to enable the model to replay the event log and accurately express the actual business process. In the existing method, the model is repaired based on differences between the event log and the model. For example, in the Fahland method, firstly, deviations between the event log and the model are obtained according to an optimal alignment and non-fitted sub-logs are collected; next, a corresponding sub-process is mined and added into an appropriate position of the original model as a self-loop, or a cycle capable of replaying the sub-log is added into the original model. The Goldratt method focuses on resources consumed for repairing the process model. In the method, the model is repaired through two types of operations of skipping one activity or adding a single activity into the original model in the form of self-loop. Although the repaired models obtained by the above two methods both have high fitness, the precision of the model is reduced due to appearance of the self-loop, resulting in inability to reflect the actual business process correctly. When a part of the business process is changed, that is, relationships between some activities in the event log cannot be described by a corresponding sub-model, the original sub-model shall be replaced with a correct sub-model. In addition, a traditional Petri net cannot express a special relationship between some activities in the log. In this case, the model obtained by the existing repairing method is complex and cannot reflect a logical relationship between activities. Therefore, the actual business process cannot be correctly expressed by the process model. The execution efficiency of the business process is obviously reduced after it is updated (or changed), thereby increasing the time of persons in handling businesses.

### SUMMARY

An object of the present disclosure is to provide a process model repairing method based on structure replacement so as to improve a repair precision of a process model and enable the obtained process model to express an actual business process correctly.

To achieve the above object, the present disclosure adopts the following technical solution.

A process model repairing method based on structure replacement includes the following steps.

At step I, two ordering relationship sets of an event log and a model are obtained by re-defining ordering relationships between activities, and a deviation set recording differences between the event log and the model is obtained.

A business process will change along with a change of an actual situation; at this time, it is required to perform conformance checking for an original process model and an event log generated by an actual business process.

If the process model is not conformant with the event log, a deviation will be discovered.

A method of collecting all deviations between the event log and the process model based on an extended ordering relationship is proposed below.

To accurately identify some relationships between activities in the event log, the following concepts are proposed.

The extended ordering relationship is defined.

It is assumed that a set A⊆ [symbol] , L∈ [symbol] (A*) is an event log, and σ∈L is a trace.
For any activities a, b∈σ; there are:

(1) a follow-up relationship >: a>b, when and only when ∃σ∈L: σ[i]=a, σ[i+1]=b, 1≤i<|σ|;

(2) a causal relationship →: a→b, when and only when ∀σ∈L, a, b∈&(σ): ∃a>b and [symbol] b>a, where &(σ) refers to a set formed by all activities in the trace σ;

(3) a selective relationship ×: a×b, when and only when ∀σ∈L: a∈&(σ) and b∈&(σ), or b∈&(σ) and a∉v&(σ);

(4) a common concurrent relationship ∥: a∥b, when and only when ∃σ₁, σ₂∈L: for a, b∈&(σ₁), a>b and for a, b∈&(σ₂), b>a; and

(5) a logical concurrent relationship ∨: a∨b, when and only when ∃σ₁, σ₂, σ₃∈L: for σ₁, σ₂∈L, a∥b, and for σ₃∈L, a∈&(σ₃) and b∉&(σ₃), or b∈&(σ₃) and a∉&(σ₃), where σ₁, σ₂, σ₃ refer to traces in an event log L.

A log ordering set is defined:

It is assumed that ⊕={→, ×, ∥, ∨} is a symbol set.

R_{L}=(a⊕b|a, b∈A) being referred to as the log ordering set, where a⊕b refers to an ordering relationship between activities a and b.

A model ordering set is defined.

It is assumed that PN=(P, T; F, M) is a Petri net, S_{PN}∈ [symbol] (T*) is a complete firing sequence set, and ⊕′={→, ×, ∥} is a symbol set; R_{M}={tᵢ⊕tⱼ|tᵢ, tⱼ∈T} is a model ordering set, where tᵢ⊕tⱼ refers to an ordering relationship between tᵢ and tⱼ based on S_{PN}.

A logical model ordering set is defined.

It is assumed that LPN=(P, T; F, I, O, M) is a logical Petri net, S_{LPN}∈ [symbol] (T*) is a complete firing sequence set, and ⊕={→, ×, |, ∧} is a symbol set; R_{LM}={tᵢ⊕tⱼ|tᵢ, tⱼ∈T} is a logical model ordering set, where tᵢ⊕tⱼ refers to an ordering relationship between tᵢ and tⱼ based on S_{LPN}.

According to the above definitions, a method of obtaining a log ordering set from the event log L is proposed below.

Method 1: a method of generating an extended ordering relationship includes the following steps:

At step 1, a complete event log L∈ [symbol] (A*) is input, and let R=Ø, R_{L}=Ø, where R refers to a set.

At step 2, for any σ∈L, aᵢ∈σ, 1≤i<|a| are satisfied; if aᵢ>aᵢ₋₁∉R, R=R∪{aᵢ>aᵢ₋₁}.

At step 3, if any element in R satisfies ∃a>b and [symbol] b>a, R_{L}=R_{L}∪{a→b}.

At step 4, if any element in R satisfies a∈&(σ) and b∉&(σ), or a∉&(σ) and b∈&(σ), R_{L}=R_{L}∪{a×b}.

At step 5, if any element in R satisfies a>b, b>a, and for any σ∈L, a, b∈&(σ), then R_{L}=R_{L}∪{a∥b}.

At step 6, if any element in R satisfies a>b, b>a, and for σ∈L, a∈&(σ) and b∈&(σ), or a∉&(σ) and b∈&(σ), then R_{L}=R_{L}∪{a∨b}, where a>b, b>a refers to that activities a and b are in concurrent relationship.

At step 7, the log ordering set R_{L} is obtained.

The log ordering set R_{L} records ordering relationships between all activities in the event log L.

According to principles of the method 1, the event log L at each step is changed into the complete firing sequence set S_{PN} to generate the model ordering set R_{M} of a Petri net.

The model ordering set R_{M} of the Petri net records relationships between all transitions in a Petri net model PN.

By comparing R_{L} and R_{M}, all deviations between the log L and the Petri net model PN can be discovered.

A deviation set is defined.

It is assumed that R_{D} is a deviation set, including

(1) R_{D}=Ø, when and only when ∃a⊕b∈R_{L} and a⊕b∈R_{M};

(2) a⊕b|a⊕′b∈R_{D}, when and only when ∃a⊕b∈R_{L} and a⊕′b∈R_{M};

(3) a⊕b|ϕ∈R_{D}, when and only when ∃a⊕b∈R_{L}; and

(4) ϕ|a⊕b∈R_{D}, when and only when ∃a⊕b∈R_{M}, where the symbol R_{D}(a) refers to the number of elements containing a in R_{D};

It can be known from the above definition that the deviation set R_{D} records different ordering relationships in the event log and the model.

Method 2: a method of generating a deviation set includes the following steps.

At step 1, the log ordering set R_{L} and the model ordering set R_{M} are input, and let the deviation set be R_{D}=Ø.

At step 2, for any a⊕b∈R_{L}, if a⊕b∈R_{M}, R_{D}=Ø.

At step 3, for any a⊕b∈R_{L}, if a⊕′b∈R_{M}, R_{D}=R_{D}∪{a⊕b|a⊕′b}.

At step 4, for any a⊕b∈R_{L}, if a⊕b∈R_{M} and a⊕′b∈R_{M} are not established, R_{D}=R_{D}∪{a⊕b|ϕ}.

At step 5, for any a⊕b∈R_{M}, if a⊕′b∈R_{L} is not established, R_{D}=R_{D}∪{ϕ|a⊕b}.

At step 6, the deviation set R_{D} between the log and the model is obtained.

At step II, the repair of the model is completed by repairing the corresponding sequential structure or selective structure into the concurrent structure based on the deviation set.

At step II.1, the repair of the model based on the selective structure includes the following steps.

At step 1, the complete event log L and the Petri net PN=(P, T F, M) are input; the repaired logical Petri net model is defined as LPN′=(P′, T′; F′, I′, O′, M′), and let LPN′=PN, R_{D}′=Ø;

At step 2, the log ordering set R_{L} and the model ordering set R_{M} are obtained by calling the method of generating an extended ordering relationship.

At step 3, the deviation set R_{D} is obtained by calling the method of generating a deviation set.

At step 4, for any a∨b|a×b∈R_{D}, if ^{●}a∩^{●}b=Ø or ^{●}a∩b^{●}=Ø, R_{D}′=R_{D}′∪{R_{D}−a∨b|a×b}.

At step 5, for any a∨b|a×b∈R_{D}′, when ^{●}a∩^{●}b=Ø, sizes of R_{D}(a) and R_{D}(b) are determined.

If R_{D}(a)≥R_{D}(b), the activity a is a deviation activity at this time; let tₒ=^{●}(^{●}a)∩^{●}(^{●}b) be a logical output transition, P′=P′∪{pₒ} and pₒ=^{●}a, F′=F′−{^{●}b→a}∪{tₒ→pₒ, pₒ→a}, O′=O′∪{O′(tₒ)=pₒ∨^{●}b}.

If R_{D}(b)≥R_{D}(a), the activity b is a deviation activity at this time; let tₒ=(^{●}a)∩^{●}(^{●}b) be a logical output transition, P′=P′∪{pₒ} and pₒ=^{●}b, F′=F′−{^{●}a→b}∪{tₒ→pₒ, pₒ→b}, O′=O′∪{O′(tₒ)=pₒ∨^{●}a}.

At step 6, for any a∨b|a×b∈R_{D}′, if a^{●}∩b^{●}≠Ø, sizes of R_{D}(a) and R_{D}(b) are determined.

If R_{D}(a)≥R_{D}(b), the activity a is a deviation activity at this time; let tᵢ=(a^{●})^{●}∩n(b^{●})^{●} be a logical input transition, P′=P′∪{pᵢ} and pᵢ=a^{●}, F′=F′−{a→b^{●}}∪{a→pᵢ, pᵢ→tᵢ}, I′=I′∪{I′(tᵢ)=pᵢ∨b^{●}}.

If R_{D}(b)≥R_{D}(a), the activity b is a deviation activity at this time; let tᵢ=(a^{●})^{●}∩(b^{●})^{●} be a logical input transition, P′=P′∪{pᵢ} and pᵢ=b^{●}, F′=F′−{b→a^{●}}∪{b→pᵢ, pᵢ→tᵢ}, I′=I′∪{I′(tᵢ)=pᵢ∨a^{●}}.

At step 7, the repaired model LPN′ based on the selective structure is obtained.

Thus, the updated business process is correctly expressed by executing the updated business process with the repaired process model.

At step II.2, the repair of the model based on the sequential structure includes the following steps.

At step 1, the complete event log L and the Petri net PN=(P, T; F, M) are input; a repaired logical Petri net model LPN″=(P″, T″; F″, I″, O″, M″) is defined; let LPN″=PN, R_{D}′=Ø;

At step 2, the log ordering set R_{L} and the model ordering set R_{M} are obtained by calling the method of generating an extended ordering relationship.

At step 3, the deviation set R_{D} is obtained by calling the method of generating a deviation set.

At step 4, for any a∨b|a→b∈R_{D}, F″=F″−{a^{●}→b}.

At step 5, for any a→b|ϕ∈R_{D}, if b∨c|ϕ∈R_{D} and a=^{♦}b∩^{♦}c, a=^{●}(^{●}c),

then P″=P″∪{pₒ}(pₒ=^{●}b), F″=F″∪{a→pₒ, pₒ→b}, O″=O″∪{O″(a)=^{●}b∨^{●}c}.

At step 6, for any a→b|ϕ∈R_{D}, if a∨c|a→c∈R_{D} and b=a^{♦}∩c^{♦}, b=(c^{●})^{●}.

then F″=F″∪{a^{●}→b}, I″=I″∪{I″(b)=a^{●}∨c^{●}}, where a, b and c refer to different activity names.

At step 7, obtaining the repaired model LPN″ based on the sequential structure;

Thus, the updated business process is correctly expressed by executing the updated business process with the repaired process model.

The present disclosure has the following advantages.

In the method of the present disclosure, by re-defining ordering relationships between activities, the ordering relationship sets of the event log and the model are compared to obtain the deviation set; then, positions of occurrence of deviations are obtained according to the deviation set, the selective structure or the sequential structure in the model is repaired into the concurrent structure, and the traditional Petri net is converted into the logical Petri net by adding a logical expression into the model. The repaired model based on the logical Petri net may replay the event log and can accurately express the relationship between activities, so that the obtained process model can correctly express the actual business process, thereby improving the execution efficiency of the actual business process.

### BRIEF DESCRIPTIONS OF THE DRAWINGS

FIG. 1 is a schematic diagram illustrating a logical Petri net model LPN₁ according to an example of the present disclosure.

FIG. 2 is a schematic diagram illustrating a Petri net model PN₁ according to an example of the present disclosure.

FIG. 3 is a schematic diagram illustrating a repaired model LPN′ obtained based on the method of the present disclosure.

FIG. 4 is a schematic diagram illustrating a Petri net model PN₂ according to an example of the present disclosure.

FIG. 5 is a schematic diagram illustrating a repaired model LPN″ obtained based on the method of the present disclosure.

FIG. 6 is a schematic diagram illustrating a treatment process model of a tumor patient.

FIG. 7 is a schematic diagram illustrating a patient treatment process model repaired by the Fahland method.

FIG. 8 is a schematic diagram illustrating a patient treatment process model repaired by the Goldratt method.

FIG. 9 is a schematic diagram illustrating a patient treatment process model repaired by a method of the present disclosure.

FIG. 10 is a graph illustrating a change curve of fitness of a method of the present disclosure compared with the Fahland method and the Goldratt method.

FIG. 11 is a graph illustrating a change curve of precision of a method of the present disclosure compared with the Fahland method and the Goldratt method.

### DETAILED DESCRIPTIONS OF EMBODIMENTS

A basic idea of the present disclosure is to provide a new model repairing method taking an event log and a model as inputs. If the model conforms to the log, it is unnecessary to repair the model. If the model cannot replay the event log, it is required to repair the model according to a conformance checking result. Unlike process discovery, a portion of the model capable of replaying the event log is reserved in repair of the model, thereby ensuring similarity between the repaired model and the original model.

The present disclosure is further described in detail below in combination with accompanying drawings and specific examples.

A Petri net can describe and analyze an information system with concurrency, asynchronization, distribution and uncertainty. A logical Petri net, as an extension of the Petri net, may improve fitness and precision of a model.

Basic definitions of the Petri net and the logical Petri net are briefly introduced below.

A process model repairing method based on structure replacement includes the following steps.

A multiset is defined.

S refers to a set, a multiset D on the set S is a mapping D: S→N₊, and N₊ refers to a set of positive integers. B(S) refers to a set of all multisets on the set S.

A trace and an event log are defined.

It is assumed that A is a set of all activities. If an activity sequence is σ∈A* with A* referring to a set of finite sequences on the set A,σ is referred to as a trace. If L∈B(A*) is a multiset of the trace, L is referred to as an event log.

&(σ) refers to a set formed by all activities in the trace σ.

An element position is defined.

Assuming v=(a₁, a₂, . . . , aₙ) is an n-tuple, where aᵢ∈S and 1≤i≤n, then v[i]=aᵢ refers to an i-th element of v.

For example, v=(a, b) and a,b∈S, then v[1]=a and v[2]=b.

A pre-activity set and a post-activity set are defined.

It is assumed that A refers to a set of all activities, L∈B(A*) is an event log, and e∈A is an activity of L;

^{♦}e={a|∀σ∈L∧a∈A∧<a, e>∈σ}, e^{♦}={a|∀σ∈L∧a∈A∧<e, a>∈σ};

^{♦}e is a pre-activity set of e, and e^{♦} is a post-activity set of e, where a refers to an activity in the trace σ.

A pre-set and a post-set are defined.

It is assumed that N=(P, T; F) is a net, where P is a set of finite places, T is a set of finite transitions, and F⊆(P×T)∪(T×P) is a set of finite arcs of the net N.

For ∀x∈P∪T, let*x={y|y∈P∪T∧(y, x)∈F}, x^{●}={y|y∈P∪T∧(x, y)∈F};

^{●}x is a pre-set or an input set of x, x^{●} is a post-set or an output set of x, where y refers to a place or a transition.

The Petri net is defined.

A quadruple PN=(P, T; F, M) is referred to as a Petri net, when and only when

(1) N=(P, T; F) is a net;

(2) a mapping is M: P→N₊ and M is referred to as a marking of the net N, where Mᵢ is an initial marking, and M_{f} is a final marking;

(3) a transition firing rule:

1) for a transition t∈T, if ∀p∈^{●}t:M(p)≥1, the transition t is enabled under the marking M, which is denoted as M [t>;

2) if M [t>, the transition t may be fired under the marking M, a new marking M′ is obtained by firing the transition t from the marking M, which is denoted as M [t>M′; for ∀p∈P, the following is established:

$${M}^{′} ( p ) = \{ \begin{matrix}M ( p ) - 1 , & p ∈ {}^{*} t - {t}^{*} \\ M ( p ) + 1 , & p ∈ {t}^{*} - {}^{*} t \\ M ( p ) , & other\end{matrix} .$$

A set of all markings reachable from M is denoted as R(M) and specified as M∈R(M).

A complete firing sequence is defined.

It is assumed that PN=(P, T; F, M) is a Petri net, and a sequence s∈T* is referred to as a complete firing sequence. When and only when Mₒ[s>M_{f}, one complete firing sequence set S_{FN}∈ [symbol] (T*) includes all firing sequences in PN.

T* refers to a set of finite sequences on the set T.

Mₒ[s>M_{f} refers to that all transitions in the sequence s may be sequentially fired under the marking Mₒ, and a final marking M_{f} is obtained.

The logical Petri net is defined.

A six-tuple LPN=(P, T; F, I, O, M) is referred to as a logical Petri net, including

(1) P is a set of finite places;

(2) T=T_{I}∪T_{O}∪T_{D} is a set of finite transitions, T∩P=Ø, T∪P≠Ø; if t∈T_{I}∩T_{O}, ^{●}t∩t^{●}=Ø, where,

1) T_{I} refers to a logical input transition set: for ∀t∈T_{I}, an input place ^{●}t of t is limited by a logical expression f_{I}(t);

2) T_{O} refers to a logical output transition set; for ∀t∈T_{O}, an output place t^{●} of t is limited by a logical expression f_{O}(t);

3) T_{D} refers to a transition set in the traditional Petri net;

(3) F⊆(P×T)∪(T×P) is a set of finite arcs;

(4) I refers to a mapping of the logical input transition to a logical input function for ∀t∈T_{I}, I(t)=f_{I}(t);

(5) O refers to a mapping of the logical output transition to a logical output function; for ∀t∈T_{O}, O(t)=F_{O}(I);

(6) M: P→{0,1} is a marking function; for ∀p∈P, M(p) refers to the number of tokens included in p;

(7) a transition firing rule:

1) for ∀t∈T_{I}, if f_{I}(t)|_{M}=_{●}T_{●}, the logical input transition t may be fired and denoted as M[t>M′; for ∀p∈^{●}t: M′(p)=0; ∀p∈t^{●}: M′(p)=1, ∀p∉t^{●}∪^{●}t: M′(p)=M(p);

2) for ∀t∈T_{O}, if f_{O}(t)|_{M}=_{●}T_{●}, the logical output transition t is enabled and denoted as M[t>M′; for ∀p∈^{●}t: M′(p)=0: ∀p∈t^{●}: M(p)=, ∀p∉t^{●}∪^{●}t: M′(p)=M(p);

3) for ∀t∈T_{D}, the transition firing rule is conformant to the traditional Petri net; and

(8) if p₁ and p₂ are pre-sets (post-sets) of the logical input (output) transition t,

1) p₁⊗p₂ refers to that when and only when one of p₁ and p₂ includes token, t is enabled; or

after t is fired, only one of p₁ and p₂ includes token;

2) p₁∨p₂ refers to that when and only when p₁ and p₂ both include token, t is enabled; or

after t is fired, p₁ and p₂ both include token; and

3) p₁∨p₂ refers to that when and only when at least one of p₁ and p₂ includes token, t is enabled; or

after t is fired, at least one of p₁ and p₂ includes token.

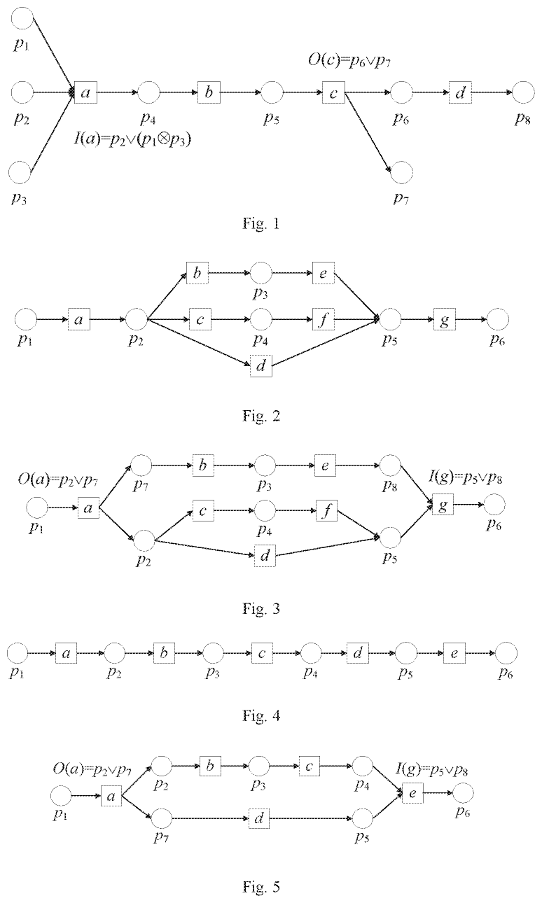

FIG. 1 illustrates a logical Petri net LPN₁.

A transition a is a logical input transition, and c is a logical output transition. Other transitions are common transitions. I(a)=p₂∨(p₁⊗p₃) is a logical input function of a and indicates that a may be enabled in the following five cases:

(1) only p₁ includes token: (2) only p₂ includes token; (3) only p₃ includes token; (4) p₁ and p₂ both include token; (5) p₁ and p₃ both include token.

O(c)=p₆∨p₇ is a logical output function of c, and indicates that at least one of p₆ and p-includes token after c is fired.

At step I, two ordering relationship sets of an event log and a model are obtained by re-defining ordering relationships between activities, and a deviation set recording differences between the event log and the model is obtained.

With a change of an actual situation, a business process may be changed to adapt to a changing environment. At this time, it is required to perform conformance checking for an original model and an event log generated in an actual process.

If the model is not conformant with the event log, a deviation is discovered.

The present disclosure provides a method of collecting all deviations between an event log and a process model based on an extended ordering relationship.

To effectively analyze the event log, it is required to extract main information of the log.

To accurately identify some relationships between activities in the event log, the following concepts are proposed.

The extended order relationship is defined.

It is assumed that a set A⊆ [symbol] , L∈ [symbol] (A*) is an even log, and σ∈L is a trace. For any a, b∈σ, there are:

(1) a follow-up relationship >: a>b, when and only when ∃σ∈L: σ[i]=a, σ[i+1]=b, 1≤i<|σ|;

(2) a causal relationship →: a→b, when and only when ∀σ∈L, a, b∈&(σ): ∃a>b and [symbol] b>a;

(3) a selective relationship ×: a×b, when and only when ∀σ∈L: a∈&(σ) and b∈&(σ), or b∈&(σ) and a∉v&(σ);

(4) a common concurrent relationship ∥: a∥b, when and only when ∃σ₁, σ₂∈L: for a, b∈&(σ₁), a>b and for a, b∈&(σ₂), b>a; and

(5) a logical concurrent relationship ∨: a∨b, when and only when ∃σ₁, σ₂, σ₃∈L: for σ₁, σ₂∈L, a∥b, and for σ₃∈L, a∈&(σ₃) and b∉&(σ₃), or b∈&(σ₃) and a∉&(σ₃).

A log ordering set is defined.

It is assumed that a set A⊆ [symbol] , L∈ [symbol] (A*) is an event log, and ⊕={→, ×, ∥, ∨} is a symbol set. R_{L}={a⊕b|a, b∈A} is referred to as the log ordering set, where a⊕b refers to an ordering relationship between activities a and b.

The ordering set R_{L} of the log L is obtained according to the definition of the log ordering set. The log ordering set is another expression form of the event log, and one log ordering set can express ordering relationships between any activities in the corresponding event log in a formalized manner.

For example, for ∀L∈ [symbol] (A*) and a, b∈A, the ordering relationship between a and b is a→b, a×b, a∥b, or a∨b, where:

a→b refers to the causal relationship, that is, b can be fired only after a is fired:

a×b refers to the selective relationship, that is, a and b cannot be fired simultaneously in the same trace;

a∥b refers to the common concurrent relationship, that is, in any trace, if a is fired, b is definitely fired:

a∨b refers to the logical concurrent relationship, that is, there are at least three traces, a∥b may be obtained from two traces, but a and b cannot be fired simultaneously in one remaining trace.

The log ordering set can be obtained according to the extended ordering relationship between event logs, and the model ordering set can be obtained according to the Petri net model.

The model ordering set is defined.

It is assumed that PN=(P, T; F, M) is a Petri net, S_{PN}∈ [symbol] (T*) is a complete firing sequence set, and ⊕={→, ×, ∥} is a symbol set. R_{M}={tᵢ⊕tⱼ|tᵢ, tⱼ∈T} is a model ordering set, where tᵢ⊕tⱼ refers to an ordering relationship between tᵢ and tⱼ based on S_{PN}.

According to the definition of the model ordering set, the model ordering set of the logical Petri net may be obtained.

The logical model ordering set is defined.

It is assumed that LPN=(P, T; F, I, O, M) is a logical Petri net. S_{LPN}∈ [symbol] (T*) is a complete firing sequence set, and ⊕={→, ×, ∥, ∨} is a symbol set.

R_{LM}={tᵢ⊕tⱼ|tᵢ, t∈T} is a logical model ordering set, where tᵢ⊕tⱼ refers to an ordering relationship between tᵢ and tⱼ based on S_{LPN}.

According to the above definitions, a method of obtaining a log ordering set from an event log is proposed below, as shown in method 1.

Method 1: a method of generating an extended ordering relationship includes the following steps.

At step 1, a complete event log L∈ [symbol] (A*) is input; a log ordering set R_{L} is output;

let R=Ø, R_{L}=Ø, where R refers to a set.

At step 2, for any σ∈L, aᵢ∈σ and 1≤i<|σ| are satisfied; if aᵢ>aᵢ₋₁∉R, R=R∪{aᵢ>aᵢ₋₁}.

At step 3, if any element in R satisfies ∃a>b and ∃/b>a, R_{L}=R_{L}∪{a→b}.

At step 4, if any element in R satisfies a∈&(σ) and b∈&(σ), or a∈&(σ) and b∈&(σ), R_{L}=R_{L}∪{a×b}.

At step 5, if any element in R satisfies a>b, b>a, and for any σ∈L, a, b∈&(σ), then R_{L}=R_{L}∪{a∥b}.

At step 6, if any element in R satisfies a>b, b>a, and for σ∈L, a∈&(σ) and b∈&(σ), or a∉&(σ) and b∈&(σ), then R_{L}=R_{L}∪{a∨b}.

At step 7, the log ordering set R_{L} is obtained.

According to principles of the method 1, the event log L at each step may be changed into the complete firing sequence set S_{PN} to generate the model ordering set R_{M} of a Petri net.

Example 1: an original process model PN₁ of a particular business process is as shown in FIG. 2.

There is an event log L₁={σ₁, σ₂, σ₃, σ₄, σ₅, σ₆, σ₇, σ₈, σ₉, σ₁₀, σ₁₁, σ₁₂}{<a, b, e, g>, <a, c, f, g>, <a, d, g>, <a, b, e, c, f, g>, <a, b, c, e, f, g>, <a, b, c, f, e, g>, <a, c, b, e, f, g>, <a, c, b, f, e, g>, <a, c, f, b, e, g>, <a, b, e, d, g>, <a, b, d, e, g>, <a, d, b, e, g>}.

Extended ordering relationships are listed below, and a log ordering set and a model ordering set are obtained.

For ∀σ∈L₁, the following extended ordering relationship may be obtained.

(1) A follow-up relationship >:

σ₁>={a>b,b>e,e>g};

σ₂>={a>c,c>f,f>g};

σ₃>={a>d,d>g};

σ₄>={a>b,b>e,e>c,c>f,f>g};

σ₅>={a>b,b>c,c>e,e>f,f>g};

σ₆>={a>b,b>c,c>f,f>e,e>g};

σ₇>={a>c,c>b,b>e,e>f,f>g};

σ₈>={a>c,c>b,b>f,f>e,e>g};

σ₉>={a>c,c>f,f>b,b>e,e>g};

σ₁₀>={a>b,b>e,e>d,d>g};

σ₁₁>={a>b,b>d,d>e,e>g};

σ₁₂>={a>d,d>b,b>e,e>g};

R={a>b a>c,a>d,b>e,b>c,c>b,b>d d>b,b>f,f>b,c>e,e>c,c>f,d>e,e>d,e>f,f>e,d>g,e>g,f>g}.

(2) A causal relationship →:

R_{L1}=R_{L1}∪{a→b,b→e,e→g,a→c,c→f,f→g,a→d,d→g}.

(3) A selective relationship ×:

R_{L1}=R_{L1}∪{c×d,d×f}.

(4) A common concurrent relationship ∥:

R_{L1}=R_{L1}∪Ø.

(5) A logical concurrent relationship ∨:

R_{L1}=R_{L1}∪{b∨c,b∨d,b∨f,c∨e,d∨e,e∨f}.

Thus, the ordering relationship set of L₁ is obtained as follows:

R_{L1}={a→b,b→e,e→g,a→c,c→f,f→g,a→d,d→g,c×d,d×f,b∨c,b∨d,b∨f,c∨e,d∨e,e∨f}.

There are three complete firing sequences in PN₁ as follows:

s₁=<a, b, e, g>, s₂=<a, c, f, g>, and s₃=<a, d, g>. All ordering relationships in PN₁ are shown as follows:

(1) a follow-up relationship >:

s₁>={a>b,b>e,e>g};

s₂>={a>c,c>f,f>g};

s₃>={a>d,d>g};

R={a>b,a>c,a>d,b>e,c>f,d>g,e>g,f>g};

(2) a causal relationship →:

R_{M1}=R_{M1}∪{a→b,b→e,e→g,a→c,c→f,f→g,a→d,d→g};

(3) a selective relationship ×:

R_{M1}=R_{M1}∪{b×c,b×d,b×f,c×d,c×e,e×f};

(4) a common concurrent relationship ∥:

R_{M1}=R_{M1}∪Ø; and

(5) a logical concurrent relationship ∨:

R_{M1}=R_{M1}∪Ø.

Thus, the model ordering set ofPNI may be obtained as follows:

R_{M1}={a→b,b→e,e→g,a→c,c→f,f→g,a→d,d→g,b×c,b×d,b×f,c×d,c×e,d×e,e×f}.

R_{L} records ordering relationships between all activities in the event log L, and R_{M} records relationships between all transitions in the Petri net model PN. By comparing R_{L} with R_{M}, all deviations between L and PN can be discovered.

To store the deviations therebetween, the present disclosure proposes a concept of a deviation set.

The deviation set is defined.

It is assumed that R_{D} is a deviation set, including

(1) R_{D}=Ø, when and only when ∃a⊕b∈R_{L} and a⊕b∈R_{M};

(2) a⊕b|a⊕′b∈R_{D}, when and only when ∃a⊕b∈R_{L} and a⊕′b∈R_{M};

(3) a⊕b|ϕ∈R_{D}, when and only when ∃a⊕b∈R_{L}; and

(4) ϕ|a⊕b∈R_{D}, when and only when ∃a⊕b∈R_{M}.

In the present disclosure, symbol R_{D}(a) refers to the number of elements containing a in R_{D}.

It can be known from the above definition that R_{D} records different ordering relationships in the event log and the model.

There are mainly three reasons that the model cannot replay the event log:

(1) Some new activities appear in the log.

(2) The relationship between original activities in the log cannot be described by a corresponding sub-model in the model.

(3) Behaviors in the model do not appear in the log.

A method of generating a deviation set is proposed below, as shown in method 2.

Method 2: a method of generating a deviation set includes the following steps.

At step 1, the log ordering set R_{L} and the model ordering set R₄ are input; the deviation set R_{D} between the log and the model is defined: let the deviation set be R₁=0.

At step 2, for any a⊕b∈R_{L}, if a⊕b∈R_{M}, R_{D}=Ø.

At step 3, for any a⊕b∈R_{L}, if a⊕b∈R_{M}, R_{D}=R_{D}∪{a⊕b|a⊕′b}.

At step 4, for any a⊕b∈R_{L}, if a⊕b∈R_{M} and a⊕′b∈R_{M} are not established, R_{D}=R_{D}∪{a⊕b|ϕ}.

At step 5, for any a⊕b∈R_{M}, if a⊕′b∈R_{L} is not established, R_{D}=R_{D}∪{ϕ|a⊕b}.

At step 6, the deviation set R_{D} is obtained.

Example 2: according to the example 1, there is R_{L}={a→b, b→e, e→g, a→c, c→f, f→g, a→d, d→g, c×d, d×f, b∨c, b∨d, b∨d, b∨f, c∨e, d∨e, e∨f} and R_{M1}={a→b, b→e, e→g, a→c, c→f, f→g, a→d, d→g, b×c, b×d, b×f, c×d, c×e, d×e, e×f}. By comparing R_{L1} with R_{M1}, the deviation set obtained by the method 2 is as follows:

R_{D1}={b∨c|b×c,b∨d|b×d,b∨f|b×f,c∨e|c×e,d∨e|d×e,e∨f|e×f}.

At step II, the repair of the model is completed by repairing the corresponding sequential structure or selective structure into the concurrent structure based on the deviation set.

With the update of the business process system, the original process model is not updated in time and thus not matched with the actual business process, thereby failing to correctly replay the new event log reflected in the process.

If the ordering relationship between some activities in the event log cannot be described by the corresponding sub-model, it is required to replace the original sub-model with a new sub-model, so that the new model may describe the relationship between activities.

For example, some activities in the original process model belong to the sequential structure or the selective structure, but these activities in the event log corresponding to the actual business process have a logical concurrent relationship. At this time, it is required to change the sub-model in the original process model according to the event log, that is, replace the sequential structure or the selective structure in the original model with the concurrent structure.

An example of the present disclosure provides two methods of changing a sub-model by constructing a concurrent structure.

The first method is to repair the model based on the selective structure.

With the change of the actual process, some activities in the event log have the logical concurrent relationship.

The activities having such relationship may be executed simultaneously, or at least one of the activities may be executed.

However, only one of the activities can be fired in the actual model, that is, these activities belong to the selective structure. At this time, the model may be repaired by changing the selective structure into the concurrent structure. All activities in the concurrent structure may be fired simultaneously in the model.

By adding a logical expression into the model, the obtained repaired model may fire at least one of the activities, thereby improving applicability of the business process. A method of repairing a model including a selective structure is proposed below, as shown in a method 3.

Method 3: a method of repairing a model based on a selective structure includes the following steps.

At step 1, the complete event log L and the Petri net PN=(P, T; F, M) are input; the repaired logical Petri net model is defined as LPN′=(P′, T′; F′, I′, O′, M′); let LPN′=PN, R_{D}′=Ø.

At step 2, R_{L} and R_{M} are obtained by calling the method of generating an extended ordering relationship (i.e., method 1).

At step 3, R_{D} is obtained by calling the method of generating a deviation set (i.e., method 2).

At step 4, for any a∨b|a×b∈R_{D}, if ^{●}a∩^{●}b=Ø or a^{●}∩b^{●}=Ø, then R_{D}′=R_{D}′∪{R_{D}−a∨b|a×b}.

At step 5, for any a∨b|a×b∈R_{D}′, if ^{●}a∩^{●}b≠Ø, sizes of R_{D}(a) and R_{D}(b) are determined.

If R_{D}(a)≥R_{D}(b), the activity a is a deviation activity at this time; let tₒ=^{●}(^{●}a)∩^{●}(^{●}b) be a logical output transition, P′=P′∪{pₒ} and pₒ=^{●}a, F′=F′−{^{●}b→a}∪{tₒ→pₒ, pₒ→a}, O′=O′∪{O′(tₒ)=pₒ∨^{●}b}.

If R_{D}(b)≥R_{D}(a), the activity b is a deviation activity at this time: let tₒ=^{●}(^{●}a)∩^{●}(^{●}b) be a logical output transition, P′=P′∪{pₒ} and pₒ=^{●}b, F′=F′−{^{●}a→b}∪{tₒ→pₒ, p→b}, O′=O′∪{O′(tₒ)=pₒ∨^{●}a}.

At step 6, for any a∨b|a×b∈R_{D}′, if a^{●}∩b^{●}≠Ø, sizes of R_{D}(a) and R_{D}(b) are determined.

If R_{D}(a)≥R_{D}(b), the activity a is a deviation activity at this time; let tᵢ=(a^{●})^{●}∩(b^{●})^{●} be a logical input transition, P′=P′∪{pᵢ} and pᵢ=a^{●}, F′=F′−{a→b^{●}}∪{a→pᵢ, pᵢ→tᵢ}, I′=I′∪{I′(tᵢ)=pᵢ∨b^{●}}.

If R_{D}(b)≥R_{D}(a), the activity b is a deviation activity at this time; let tᵢ=(a^{●})^{●}∩(b^{●})^{●} be a logical input transition, P′=P′∪{pᵢ} and pᵢ=b^{●}, F′=F′−{b→a^{●}}∪{b→pᵢ, pᵢ→tᵢ}, I′∪{I′(tᵢ)=pᵢ∨a^{●}}.

At step 7, the repaired model LPN′ based on the selective structure is obtained.

Thus, the updated business process can be correctly expressed by executing the updated business process with the repaired process model.

Example 3: an original process model PN₁ of a particular business process is as shown in FIG. 2.

There is a log L₁={σ₁, σ₂, σ₃, σ₄, σ₅, σ₆, σ₇, σ₈, σ₉, σ₁₀, σ₁₁, σ₁₂}={<a, b, e, g>, <a, c, f, g>, <a, d, g>, <a, b, e, c, f, g>, <a, b, c, e, f, g>, <a, b, c, f, e, g>, <a, c, b, e, f, g>, <a, c, b, f, e, g>, <a, c, f, b, e, g>, <a, b, e, d, g>, <a, b, d, e, g>, <a, d, b, e, g>}.

A process of repairing the model according to the method 3 is as follow:

It can be known from the method 3 that R_{D1}={b∨c|b×c, b∨d|b×d, b∨f|b×f, c∨e|c×e, d∨e|d×e, e∨f|e×f}, and R_{D1}′={b∨c|b×c, b∨d|b×d, d∨e|d×e, e∨f|e×f}. For b∨c|b×c∈R_{D1}′, ^{●}b∩^{●}c=p₂≠Ø. Since R_{D1}(b)>R_{D1}(c), t₁=b and t₂=c. At this time, tₒ=^{●}(^{●}t₁)∩^{●}(^{●}t₂)=a is taken as a logical output transition. Anew place p₇ is added as a pre-set of b into P. Next, an arc between p₂ and b is deleted, and two arcs from a to p and frompy to b are added respectively.

According to the existing mining algorithm, a logical output expression of a transition a is obtained as O′(a)=p₂∨p₇. For d∨e|d×e∈R_{D1}′, e^{●}∩f^{●}=p₅≠Ø. Since R_{D1}(e)>R_{D1}(d), t₁=e and t₂=d. At this time, tᵢ=(t₁^{●})^{●}∩(t₂^{●})^{●}=g is a logical input transition. A new place pgis added as a post-set of e into P. Next, an arc between e and p₅ is deleted, and two arcs from e to p₈ and from p₈ to g are added respectively. According to the existing mining algorithm, a logical input expression of a transition g is obtained as I′(g)=p₅∨p₈. The repaired model can replay all traces, and a repair result is as shown in FIG. 3.

The second method is to repair the model based on the sequential structure.

Activities sequentially executed in the actual process have a logical concurrent relationship in the event log. That is, these activities may not only be executed sequentially, but also be executed concurrently. Therefore, it is required to change the sequential structure in the original model into the concurrent structure to improve the execution efficiency of the business process.

A method of repairing a model including the sequential structure is proposed below, as shown in method 4.

Method 4: a method of repairing a model based on the sequential structure includes the following steps.

At step 1, the complete event log L and the Petri net PN=(P, T; F, M) are input; a repaired logical Petri net model LPN″=(P″, T″; F″, I″, O″, M″) is defined; let LPN″=PN, R_{D}′=Ø.

At step 2, R_{L} and R_{M} are obtained by calling the method of generating an extended ordering relationship (method 1).

At step 3 R_{D} is obtained by calling the method of generating a deviation set (method 2).

At step 4, for any a∨b|a→b∈R_{D}, F″=F″−{a^{●}→b}.

At step 5, for any a→b|ϕ∈R_{D}, if b∨c|ϕ∈R_{D} and a=^{♦}b∩^{♦}c, a=^{●}(^{●}c), then P″=P″∪{pₒ}(pₒ=^{●}b), F″=F″∪{a→pₒ, pₒ→b}, O″=O″∪{O″(a)=^{●}b∨^{●}c}.

At step 6, for any a→b|ϕ∈R_{D}, if a∨c|a→c∈R_{D} and b=a^{♦}∩c^{♦}, b=(c^{●})^{●}, then F″=F″∪{a^{●}→b}, I″=I″∪{I″(b)=a^{●}∨c^{●}}.

At step 7, the repaired model LPN″ based on the sequential structure is obtained.

Thus, the updated business process can be correctly expressed by executing the updated business process with the repaired process model.

Example 4: an original process model PN₂ of a particular business process is as shown in FIG. 4. There is an event L₂={σ₁, σ₂, σ₃, σ₄, σ₅}={<a, b, c, d, e>, <a, b, c, e>, <a, d, e>, <a, d, b, c, e>, <a, b, d, c, e>}. R_{L2}={a→b, a→d, b→c, c→e, d→e, b∨d, c∨d} and R_{M2}={a→b, b→c, c→d, d→e} may be obtained by the method 1, and R_{D}2={a→d|ϕ, c→e|ϕ, b∨d|ϕ, c∨d|c→d} may be known by the method 2. For c∨d|c→d∈R_{D2}, it is required to delete an arc from c^{●} to d. For a→d|ϕ∈R_{D2}, it may be found that b∨d also belongs to R_{D2}. At this time, a is a common pre-activity set of both b and d, and a shall be considered as a logical output transition. The new place p₇ is added as a unique pre-set of d into the model, and two arcs from a to p₇ and from p₇ to d are added simultaneously. It can be known from existing literatures that a logical output expression of a is O″(a)=^{●}b∨^{●}d=p₂∨p₇. For c→e|ϕ∈R_{D2}, c∨d|c→d∈R_{D2}. It can be known from L₂ that e is a common post-activity set of both c and d; at the same time, e is a unique post-set of d^{●} in PN₂, thus e shall be considered as a logical input transition. At this time, it is required to add an arc from c^{●} to e and calculate a logical input expression of e as I″(e)=c^{●}∨d^{●}=p₄∨p₅. The finally obtained repaired model is as shown in FIG. 5.

Experimental comparison and analysis are performed for the method of the present disclosure and both of the Fahland method and the Goldratt method.

In the present disclosure, the Fahland method is verified in a process mining tool ProM6.6, the Goldratt method is verified in a DOS window, and the repairing method in the present disclosure is manual simulation.

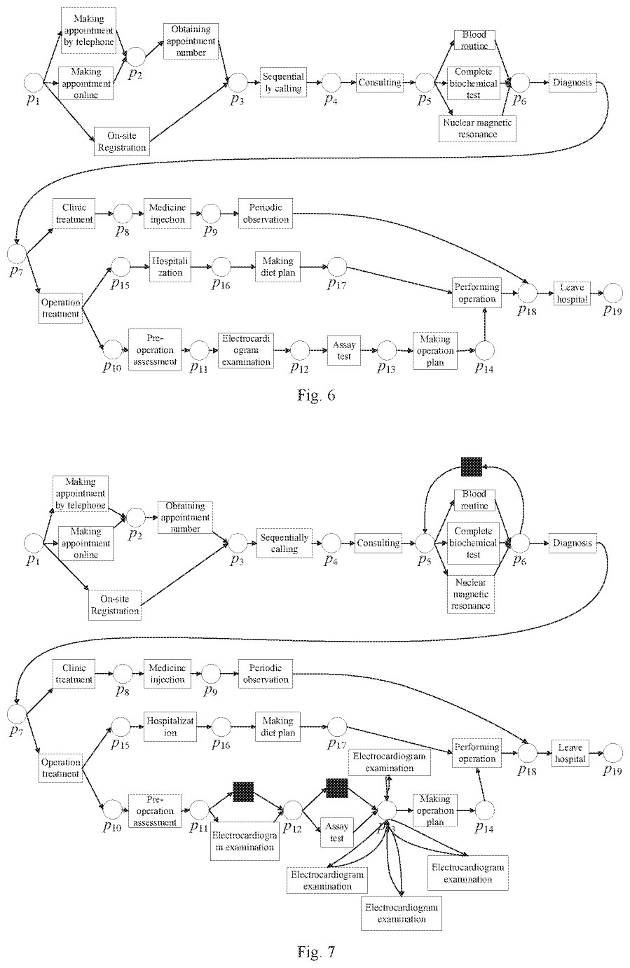

For example, in a business process of an oncology department in a particular hospital, the process model shown in FIG. 6 is obtained based on the event log generated in the original business process. A main activity process is described below.

Firstly, before going to the hospital, a patient may make an appointment with a doctor by telephone or online to obtain an appointment number: the patient may also go directly to the hospital for on-site registration rather than make an appointment. The hospital will call the appointment and registration numbers of the patients with appointment and the patients with registration in sequence, and the patients consult their doctors in sequence. After inquiring a condition of a patient, a doctor decides which examination is to be performed for the patient. There are mainly three types of examinations: blood routine, complete biochemical tests and nuclear magnetic resonance. In a diagnostic process, the doctor makes a corresponding treatment plan according to examination results and the condition of the patient. The patient with a benign tumor may receive medical treatment in an outpatient department according to the prescriptions of the doctor and can leave the hospital after a period of treatment and observation. The patient with a non-benign tumor needs to receive operation and therefore the patient should handle hospitalization procedures and make a corresponding diet plan: before operation, the doctor will make a pre-operation assessment. For this purpose, the patient takes an electrocardiogram examination and an assay test sequentially. Then, according to the examination results, the doctor makes a detailed operation plan for the patient before performing the operation. After operation, the patient may leave the hospital if the condition of the patient is improved.

With the change of the actual medical business process, some new event logs appear, and the original process model cannot reflect the actual medical business process accurately, that is, the original model cannot replay these event logs correctly. In other words, the ordering relationships between some activities in the event log cannot be described by the corresponding sub-model in the model, and thus need to be changed.

For example, in a case of examination, a patient may have more options: the patient may take blood routine and nuclear magnetic resonance, or take complete biochemical tests and nuclear magnetic resonance. In addition, before the preoperative assessment is performed, the patient may also take the electrocardiogram examination after the assay test, or only take one of them.

In the above treatment process, since the traditional Petri net cannot express the logical relationship between activities correctly, it is required to repair the model by using the logical Petri net to enable the repaired model to reflect the actual business process correctly, thereby improving the execution efficiency of the business process. In this example, the execution efficiency of the business process of the oncology department in the hospital is improved, thereby shortening time of the patient in handling relevant businesses.

Of course, the business process in the example is not limited to the above business process of the hospital, and may also be business processes of a power business hall, a mobile business hall and a bank business, and the like.

The process model matched with the above business process is repaired by the method of the present disclosure, so that the repaired process model can reflect the updated (changed) actual business process correctly, thereby improving the execution efficiency of the business process and shortening the time of persons in handling relevant businesses.

According to the event logs obtained from a hospital system, 20 groups of event logs (L₁-L₂₀) are obtained by manually removing cases that obviously deviate from the model, and these event logs include all changes mentioned above.

Table 1 gives the main attributes of these event logs, including the number of traces, the number of events, the number of activities and a length range of traces. It may be found from Table 1 that the number range of traces in the event log is 117-3215.

TABLE 1
Detailed information of 20 groups of event logs

| Event | Number of | Number of | Number of | Length |
|---|---|---|---|---|
| log | traces | events | activities | range |
| L₁ | 117 | 1667 | 22 | 9-16 |
| L₂ | 233 | 3390 | 22 | 9-16 |
| L₃ | 319 | 4728 | 22 | 9-16 |
| L₄ | 421 | 6196 | 22 | 9-16 |
| L₅ | 516 | 7373 | 22 | 9-16 |
| L₆ | 615 | 9138 | 22 | 9-16 |
| L₇ | 734 | 10716 | 22 | 9-16 |
| L₈ | 825 | 11991 | 22 | 9-16 |
| L₉ | 906 | 13308 | 22 | 9-16 |
| L₁₀ | 1138 | 16473 | 22 | 9-16 |
| L₁₁ | 1396 | 20306 | 22 | 9-16 |
| L₁₂ | 1527 | 22348 | 22 | 9-16 |
| L₁₃ | 1741 | 25650 | 22 | 9-16 |
| L₁₄ | 1913 | 28189 | 22 | 9-16 |
| L₁₅ | 2138 | 31444 | 22 | 9-16 |
| L₁₆ | 2347 | 34488 | 22 | 9-16 |
| L₁₇ | 2566 | 37497 | 22 | 9-16 |
| L₁₈ | 2708 | 39452 | 22 | 9-16 |
| L₁₉ | 3092 | 45139 | 22 | 9-16 |
| L₂₀ | 3215 | 471118 | 22 | 9-16 |

The original process model in FIG. 6 is repaired by the Fahland method, the Goldratt method and the method based on logical Petri net respectively. The number of traces included in the event log L₂₀ in Table 1 is the largest, and thus may include the most comprehensive cases.

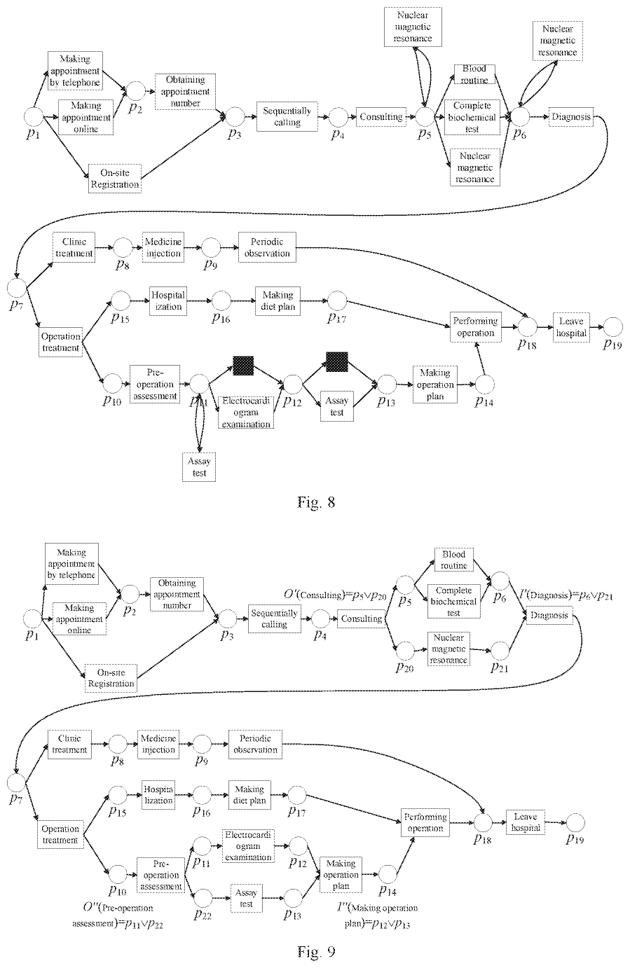

Therefore, the original model is repaired with L₂₀ as experimental data. FIGS. 7-9 illustrate repaired models obtained by these three methods respectively. All the models repaired by these methods can replay all traces in the event log L₂₀.

The model repaired by the Fahland method is as shown in FIG. 7. Firstly, deviations are obtained according to an optimal alignment between the event log and the original model, and log actions occurring at a same position are collected to construct a non-fitted sub-log. Then, a corresponding sub-process is mined and added into the model as a self-loop, or a cycle capable of replaying the sub-log is added into the original model. For model actions under alignment, invisible transition is added into the original model according to the method. Compared with the original process model in FIG. 6, the model repaired by the Fahland method is added with lcycle return transition, 2 invisible transitions, 4 repetitive transitions and 14 arcs.

The model repaired by the Goldratt is as shown in FIG. 8. The maximum fitness is sought while controlling a change amount by allocating costs and setting a maximum budget for each repair operation. The repair of the model is completed by adding the invisible transition or a single transition into the original model in the form of self-loop. Compared with the original process model in FIG. 6, the model repaired by the Goldratt method is added with 3 repetitive transitions, 2 invisible transitions and 10 arcs.

The model obtained by the method of repairing a model based on a logical Petri net in the present disclosure is as shown in FIG. 9. Compared with the original process model, the repaired model is only added with 3 places and 4 arcs. According to a logical-transition mining algorithm, a logical input function is obtained as I′(consultation)=p₆∨p₂₁ and I″(preoperative assessment)=p₁₂∨p₁₃, and a logical output function is obtained as O′(diagnosis)=^{●}t₇∨^{●}t₉=p₅∨p₂₀ and O″(operation plan making)=p₁₅∨p₂₂. The invisible transition and the repetitive transition are replaced with the logical expressions, thereby reducing complexity of the model and correctly expressing the logical relationship between activities, which cannot be obtained by the traditional Petri net.

After the process model is repaired, the method of the present disclosure is compared with two existing methods. Comparison analysis is mainly performed from three aspects of fitness, precision and simplicity according to a conformance checking rule. In the present disclosure, the fitness and the precision of each repaired model are calculated by using different quantities of event logs (as shown in Table 1).

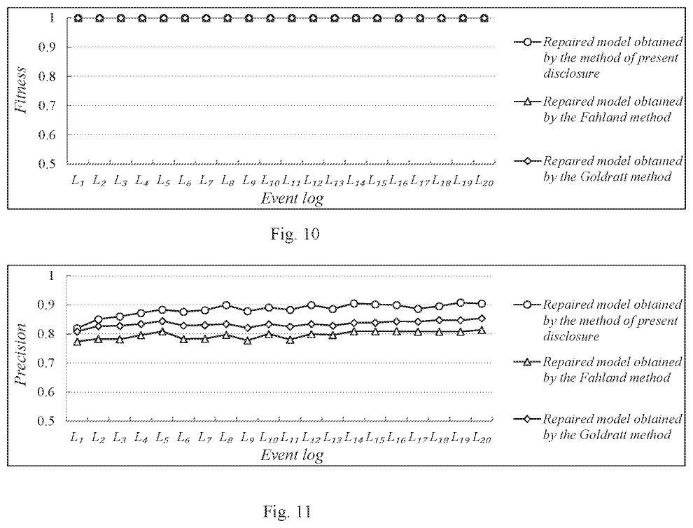

FIG. 10 is a diagram illustrating comparison results of fitnesses according to three methods. Fitness is the most important indicator for evaluating quality of the model. If all traces in the event log can be replayed in the model, a fitness value of the model is 1.

It can be seen from FIG. 10 that the fitness value of each method is always 1 under different quantities of event logs. That is, the model repaired by three methods may replay all event logs.

FIG. 11 is a diagram illustrating comparison results of precisions according to three methods. A higher precision value of the model means fewer types of other traces generated by the model except for the traces given in the event log.

It can be seen from FIG. 11 that the precision value of the model repaired by the method of the present disclosure is higher than those of other two methods. The models repaired by the Fahland method and the Goldratt method have self-loops, repetitive transitions and invisible transitions, which reduce the precision. Thus, the models cannot express the actual medical business process correctly, thereby resulting in the low execution efficiency of the actual business process.

Simplicity means that the model capable of replaying the event log shall be as simple as possible. The simplicities of three repaired models are compared according to the following criteria: the number of added places, the number of added transitions, the number of added invisible transitions and the number of added arcs.

Compared with the original model in FIG. 10, the following results are obtained from Table 2.

The repaired model obtained by the Fahland method is added with 4 transitions, 3 invisible transitions and 14 arcs, and the repaired model obtained by the Goldratt method is added with 3 transitions, 2 invisible transitions and 10 arcs; the repaired model obtained by the method of the present disclosure is added with 3 places and 4 arcs, reflecting the actual medical business process correctly.

TABLE 2
Comparison analysis of simplicities

| | | | Number | |
|---|---|---|---|---|
| | Number | Number | of added | Number |
| | of added | of added | invisible | of added |
| Model | places | transitions | transitions | arcs |
| Repaired model obtained | 3 | 0 | 0 | 4 |
| by the method of the | | | | |
| present disclosure | | | | |
| Repaired model obtained | 0 | 4 | 3 | 14 |
| by the Fahland method | | | | |
| Repaired model obtained | 0 | 3 | 2 | 10 |
| by the Goldratt method | | | | |

It can be seen from Table 2 that the simplicity of the method of the present disclosure is obviously improved compared with the Fahland method and the Goldratt method.

In conclusion, the process model obtained by the repairing method of the present disclosure can replay the event logs and accurately express the relationship between activities, thereby expressing the actual business process correctly and improving the execution efficiency of the actual business process.

Of course, the above descriptions are merely preferred examples of the present disclosure, and the present disclosure is not limited to the examples listed above. It is to be noted that all equivalent substitutions and obvious modifications made by any person skilled in the art under teaching of the present specification shall fall into the substantive scope of the present specification and shall be protected by the present disclosure.

1. A process model repairing method based on structure replacement, comprising the following steps:
at step I, obtaining two ordering relationship sets of an event log and a model by re-defining ordering relationships between activities, and obtaining a deviation set recording differences between the event log and the model,
wherein a business process changes along with a change of an actual situation; at this time, it is required to perform conformance checking for an original process model and an event log generated by an actual business process;
when the process model is not conformant with the event log, a deviation is discovered;
a method of collecting all deviations between an event log and a process model based on an extended ordering relationship is proposed below;
to accurately identify some relationships between activities in the event log, the following concepts are proposed:
defining the extended ordering relationship:
assuming that a set A⊆ [symbol] , L∈ [symbol] (A*) is an event log, and σ∈L is a trace;
for any activities a, b∈σ; there are:
(1) a follow-up relationship >: a>b, when and only when ∃σ∈L: σ[i]=a, σ[i+1]=b, 1≤i<|σ|;
(2) a causal relationship →: a→b, when and only when ∀σ∈L, a, b∈&(σ): ∃a>b and ∃/b>a,
wherein &(σ) refers to a set formed by all activities in the trace σ;
(3) a selective relationship ×: a×b, when and only when ∀σ∈L: a∈&(σ) and b∈&(σ), or b∈&(σ) and a∉v&(σ);
(4) a common concurrent relationship ∥: a∥b, when and only when ∃σ₁, σ₂∈L: for a, b∈&(σ₁), a>b and for a, b∈&(σ₂), b>a; and
(5) a logical concurrent relationship ∨: a∨b, when and only when ∃σ₁, σ₂, σ₃∈L: for σ₁, σ₂∈L, a∥b, and for σ₃∈L, a∈&(σ₃) and b∉&(σ₃), or b∈&(σ₃) and a∉&(σ₃), wherein σ₁, σ₂, σ₃ refer to traces in an event log L;
defining a log ordering set:
assuming that ⊕={→, ×, ∥, ∨} is a symbol set;
R_{L}=(a⊕b|a, b∈A) being referred to as the log ordering set, wherein a⊕b refers to an ordering relationship between activities a and b;
defining a model ordering set:
assuming that PN=(P, T; F, M) is a Petri net, S_{PN}∈ [symbol] (T*) is a complete firing sequence set, and ⊕′={→, ×, ∥} is a symbol set; R_{M}={tᵢ₁⊕′tⱼ₁|tᵢ₁|tᵢ₁, tⱼ₁∈T} is a model ordering set,
wherein tᵢ₁⊕′tⱼ₁ refers to an ordering relationship between tᵢ₁ and tⱼ₁ based on S_{PN}; and
defining a logical model ordering set:
assuming that LPN=(P, T; F, I, O, M) is a logical Petri net, S_{LPN}∈ [symbol] (T*) is a complete firing sequence set, and ⊕={→, ×, |, ∧} is a symbol set; R_{LM}={tᵢ₂⊕tⱼ₂|tᵢ₂, tⱼ₂∈T} is a logical model ordering set,
wherein tᵢ₂⊕tⱼ₂ refers to an ordering relationship between tᵢ₂ and tⱼ₂ based on S_{LPN};
according to the above definitions, a method of obtaining a log ordering set from the event log L is provided as follows:
method 1: a method of generating an extended ordering relationship, comprising:
at step 1, inputting a complete event log L∈ [symbol] (A*), letting R=Ø, R_{L}=Ø, wherein R refers to a set;
at step 2, for any σ∈L, satisfying aᵢ∈σ, 1≤i<|a|; when aᵢ>aᵢ₋₁∉R, R=R∪{aᵢ>aᵢ₋₁};
at step 3, when any element in R satisfies ∃a>b and [symbol] b>a, R_{L}=R_{L}∪{a→b};
at step 4, when any element in R satisfies a∈&(σ) and b∉&(σ), or a∉&(σ) and b∈&(σ), R_{L}=R_{L}∪{a×b};
at step 5, when any element in R satisfies a>b, b>a, and for any σ∈L, a, b∈&(σ), then R_{L}=R_{L}∪{a∥b};
at step 6, when any element in R satisfies a>b, b>a, and for σ∈L, a∈&(σ) and b∈&(σ), or a∉&(σ) and b∈&(σ), then R_{L}=R_{L}∪{a∨b},
wherein a>b, b>a refers to that activities a and b are in concurrent relationship; and
at step 7, obtaining the log ordering set R_{L},
wherein the log ordering set R_{L} records ordering relationships between all activities in the event log L;
according to the principle of the method 1, the event log L at each step is changed into the complete firing sequence set S_{PN} to generate the model ordering set R_{M} of a Petri net,
wherein the model ordering set R_{M} of the Petri net records relationships between all transitions in a Petri net model PN;
all deviations between the log L and the Petri net model PN are discovered by comparing R_{L} and R_{M};
defining a deviation set:
assuming that R_{D} is a deviation set, including
(1) R_{D}=Ø, when and only when ∃a⊕b∈R_{L} and a⊕b∈R_{M};
(2) a⊕b|a⊕′b∈R_{D}, when and only when ∃a⊕b∈R_{L} and a⊕′b∈R_{M};
(3) a⊕b|ϕ∈R_{D}, when and only when ∃a⊕b∈R_{L}; and
(4) ϕ|a⊕b∈R_{D}, when and only when ∃a⊕b∈R_{M},
wherein symbol R_{D}(a) refers to the number of elements containing a in R_{D};
it is known from the above definition that the deviation set R_{D} records different ordering relationships in the event log and the model;
method 2: a method of generating a deviation set, comprising:
at step 1, inputting the log ordering set R_{L} and the model ordering set R_{M}, and letting the deviation set be R_{D}=Ø;
at step 2, for any a⊕b∈R_{L}, when a⊕b∈R_{M}, R_{D}=Ø;
at step 3, for any a⊕b∈R_{L}, when a⊕′b∈R_{M}, R_{D}=R_{D}∪{a⊕b|a⊕′b};
at step 4, for any a⊕b∈R_{L}, when a⊕b∈R_{M} and a⊕′b∈R_{M} are not established, R_{D}=R_{D}∪{a⊕b|ϕ};
at step 5, for any a⊕b∈R_{M}, when a⊕′b∈R_{L} is not established, R_{D}=R_{D}∪{#a⊕b}; and
at step 6, obtaining the deviation set R_{D} between the log and the model; and
at step II, completing repairing the model by repairing the corresponding sequential structure or selective structure into the concurrent structure based on the deviation set,
wherein the original process model fails to be updated in time along with an update of the business process, so that the original process model is not matched with the actual business process and cannot replay a new event log that is reflected in the updated business process correctly;
therefore, it is required to repair the original process model so as to match the updated business process;
at step II.1, repairing the model based on the selective structure comprises:
at step 1, inputting the complete event log L and the Petri net PN=(P, T F, M); defining a repaired logical Petri net model LPN′=(P′, T′; F′, I′, O′, M′), and letting LPN′=PN, R_{D}′=Ø;
at step 2, obtaining the log ordering set R_{L} and the model ordering set R_{M} by calling the method of generating an extended ordering relationship;
at step 3, obtaining the deviation set R_{D} by calling the method of generating a deviation set;
at step 4, for any a∨b|a×b∈R_{D}, when ^{●}a∩^{●}b=Ø or ^{●}a∩^{●}b=Ø, R_{D}′=R_{D}′∪{R_{D}−a∨b|a×b};
at step 5, for any a∨b|a×b∈R_{D}′, when ^{●}a∩^{●}b=Ø, determining sizes of R_{D}(a) and R_{D}(b);
wherein when R_{D}(a)≥R_{D}(b), the activity a is a deviation activity at this time; let tₒ=^{●}(^{●}a)∩^{●}(^{●}b) be a logical output transition, P′=P′∪{pₒ} and pₒ=^{●}a, F′=F′−{^{●}b→a}∪{tₒ→pₒ, pₒ→a}, O′=O′∪{O′(tₒ)=pₒ∨^{●}b};
when R_{D}(b)≥R_{D}(a), the activity b is a deviation activity at this time; let tₒ=(^{●}a)∩^{●}(^{●}b) be a logical output transition, P′=P′∪{pₒ} and pₒ=^{●}b, F′=F′−{^{●}a→b}∪{tₒ→pₒ, pₒ→b}, O′=O′∪{O′(tₒ)=pₒ∨^{●}a};
at step 6, for any a∨b|a×b∈R_{D}′, when a^{●}∩b^{●}≠Ø, determining sizes of R_{D}(a) and R_{D}(b),
wherein when R_{D}(a)≥R_{D}(b), the activity a is a deviation activity at this time; let tᵢ=(a^{●})^{●}∩(b^{●})^{●} be a logical input transition, P′=P′∪{pᵢ} and pᵢ=a^{●}, F′=F′−{a→b^{●}}∪{a→pᵢ, pᵢ→tᵢ}, I′=I′∪{I′(tᵢ)=pᵢ∨b^{●}};
when R_{D}(b)≥R_{D}(a), the activity b is a deviation activity at this time; let tᵢ=(a^{●})^{●}∩(b^{●})^{●} be a logical input transition, P′=P′∪{pᵢ} and pᵢ=b^{●}, F′=F′−{b→a^{●}}∪{b→pᵢ, pᵢ→tᵢ}, I′=I′∪{I′(tᵢ)=pᵢ∨a^{●}}; and
at step 7, obtaining the repaired model LPN′ based on the selective structure;
thus, the updated business process is correctly expressed by executing the updated business process with the repaired process model; and
at step II.2, repairing the model based on the sequential structure comprises:
at step 1, inputting the complete event log L and the Petri net PN=(P, T; F, M); defining a repaired logical Petri net model LPN″=(P″, T″; F″, I″, O″, M″), and letting LPN″=PN, R_{D}′=Ø;
at step 2, obtaining the log ordering set R_{L} and the model ordering set R_{M} by calling the method of generating an extended ordering relationship;
at step 3, obtaining the deviation set R_{D} by calling the method of generating a deviation set;
at step 4, for any a∨b|a→b∈R_{D}, F″=F″−{a^{●}→b};
at step 5, for any a→b|ϕ∈R_{D}, when b∨c|ϕ∈R_{D} and a=^{♦}b∩^{♦}c, a=^{●}(^{●}c),
then P″=P″∪{pₒ}(pₒ=^{●}b), F″=F″∪{a→pₒ, pₒ→b}, O″=O″∪{O″(a)=^{●}b∨^{●}c};
at step 6, for any a→b|ϕ∈R_{D}, when a∨c|a→c∈R_{D} and b=a^{♦}∩c^{♦}, b=(c^{●})^{●}, then F″=F″∪{a^{●}→b}, I″=I″∪{I″(b)=a^{●}∨c^{●}},
wherein a, b and c refer to different activity names; and
at step 7, obtaining the repaired model LPN″ based on the sequential structure;
thus, the updated business process is correctly expressed by executing the updated business process with the repaired process model.
